(19) United States
(12) Patent Application Publication
(10) Pub. No.: US 2015/0038881 A1
(43) Pub. Date: Feb. 5, 2015

(54) POSTURE MONITORING SYSTEM

(71) Applicants: Esther Gokhale, Stanford, CA (US); Mark Leavitt, North Plains, OR (US)

(72) Inventors: Esther Gokhale, Stanford, CA (US); Mark Leavitt, North Plains, OR (US)

(21) Appl. No.: 13/987,537

(22) Filed: Aug. 5, 2013

(51) Int. Cl. A61B 5/11; A61B 5/00

(52) U.S. Cl. CPC A61B 5/1116; A61B 5/1121; A61B 5/1128; A61B 5/6805; A61B 5/6807; A61B 5/6891; A61B 5/742; A61B 5/0059
USPC 600/595

(57) ABSTRACT

A posture monitoring system includes one or more contact sensors that are used to map contact or pressure points of a person's body and produce output signals representative of the person's posture. The contact sensors are embedded within a pad, a cushion, an article of furniture or are integrated into a wearable clothing article. In accordance with the embodiments of the invention, a contact sensor is formed by depositing at least one set of flex sensors on opposite sides of a flexible substrate and electrically coupling the opposed set of flex sensors to provide a voltage bridge that cancels noise and to provide a contact sensor that measures both concave and convex changes in curvature.

### RELATED APPLICATIONS

This application claims priority under 35 U.S.C. §119(e) from the Co-pending U.S. Provisional Patent Application Ser. No. 61/850,950, filed on Feb. 28, 2013, and titled “POSTURE DETECTION SYSTEM”, the contents of which are hereby incorporated by reference.

### FIELD OF THE INVENTION

This invention relates generally to ergonomic systems and devices. More specifically, this invention relates to ergonomic systems and devices for detecting or monitoring posture using contact sensors.

### BACKGROUND OF THE INVENTION

Most people spend many hours sitting, during commutes, work, and leisure time. The way they sit and the long hours of sitting compromise their discs and other spinal elements, contributing to degenerative disc disease and the formation of osteophytes, muscular tension, and other pathological conditions.

Achieving “good” proper posture does not just refer to sitting or standing properly, but also refers to walking, sleeping, bending and lifting properly. Incorrect or “poor” posture can lead to a number health problems, including back pain. Other health problems resulting from poor posture can include spine injuries, joint problems, fatigue, muscle weakness, breathing problems, digestive problems and fatigue, to name a few.

Achieving and maintaining good or proper postures often requires changing behavior, habits and lifestyle. In order to achieve this goal, it is useful to have a continuous posture detecting or monitoring system to indicates when a person is conformal to good or proper posture and/or when a person is expressing poor or improper posture.

Prior art posture detecting or monitoring systems use accelerometers to detect or monitor a person's body position and provide an audio signal or alarm to communicate to the person when his or her posture is poor or improper. For example, U.S. Publication No. 2006/0195051 and 2009/0054814, both to Schnapp et al., describe a posture device that includes tilt sensors, an alarm, and a recording device to track changes in posture.

### SUMMARY OF THE INVENTION

The present invention is directed to a system for monitoring posture. The system includes one or more sensor structures. Sensor structures include one or more contact sensors that are responsive to structural or geometrical deformations. Preferably, the sensor structures includes one or more contact sensors that have an electrical property that changes in response to structural or geometrical deformations.

In accordance with the embodiments of the invention a sensor structure includes multiple contact sensors in a spatial arrangement. The spatial arrangement of the contact sensors allows the system to map contact or pressure points on a person's body. Changes in the electrical property of the contact sensors is measured and used to generate a graphical representation of the person's posture. In addition to the contact sensors, embodiments of the invention include optical sensors (cameras) and/or accelerometers.

Contact sensors include, but are not limited to, flex sensors, stretch sensors and pressure sensors. Flex sensors, also known as bend sensors, are uni-directional and bi-directional. In general all flex sensors change resistance when bent or deformed in one or more directions. One type of flex sensor uses a carbon-bases resistive material laminated with copper foil contacts, such as described in U.S. Pat. No. 5,411,789 to Margolin. Other flex sensors use dyes and/or polymers as resistive materials. Flex sensors have been used in robotics, gaming gloves, bio-metrics and in the automotive industry. Stretch sensors are similar flex sensor, in that they include a conductive material that changes resistance when pulled or stretched.

Pressure sensors usually include a piezo-resistive materials. For example, a pressure sensor includes a film with a carbon-impregnated polyolefin fiber that is laminated between a highly conductive, thin and flexible textile, such as Copper-polyester taffeta fabric and/or a Nickel-copper shielding material. These conductive materials can also serve as contacts for measuring changes in conductance or resistivity of the pressure sensors.

In accordance with an embodiment of the invention, a sensor structure includes one or more flex sensors or bend sensors located on opposed sides of a sensor structure and are preferably juxtaposed to one another. Theses opposed flex sensors are wired to a voltage divider that creates a balance bridge to reduce measurement errors caused by temperature changes and humidity changes. Further, the opposed flex sensor configuration described in detail below is capable of monitoring both concave and convex changes in curvature. The sensor structure is formed by depositing at least one set of flex sensors on opposite sides of a flexible substrate and electrically coupling the opposed sets of flex sensor to provide a voltage bridge that cancels noise and provide a contact sensor that measures both concave and convex changes in curvature.

In accordance with the embodiments of the invention the sensor structure is electrically coupled to detecting unit. The detecting unit includes a circuit for measuring or monitoring changes in conductance or resistance of the contact sensors. The detecting unit generates electrical signals corresponding to structural or geometrical deformations in the contact sensors. The detecting unit includes, for example, a wireless transmitter and/or an electrical connection that allows the detecting unit to communicate with a computing unit. Where the detecting unit includes a wireless transmitter, the transmitter is preferably a radio transmitter that transmits signals over remote network or is a radio transmitter that transmits signals over a peer-to peer network, such as bluetooth network. The computing unit is, for example, a personal commuter, a smart phone, an on-board vehicle computer, or other computing device with a micro-processor and memory.

The computing unit is configured to run software or firm-ware that processes or analyzes the electrical signals to general output signals that correspond to the posture of a portion of a persons's body that is in physical communication with the sensor structure. The output signals include for example audio signals, tactile signals (vibrations) and/or visual signals (graphical representations of the posture).

In accordance with the embodiments of the invention, a sensor structure, such as described above, is imbedded within a cushion structure. For example, the sensor structure is embedded within a portable cushion that is configured to be attached to a back of a chair and/or car seat. Alternatively, the sensor structure is embedded within a fixed cushion of a chair.

In accordance with still further embodiments of the invention a system includes a sensor structure with multiple sensing zones that is integrated into wearable article, such as an article of clothing. For example, a sensor structure is integrated into a belt, a vest, a band, a belt, a brace, sportswear and/or footwear. Where the sensor structure is integrated into a wearable article, the system can include one or more accelerometers to determine positions of a person's body and provide addition feed-back related to a person's posture.

In accordance with the method of the invention a sensor structure monitors a portion of a pelvic girdle of a person sitting. In operation, the sensor structure monitors a distance between the Ischium protrusion of the pelvis while a person is sitting. In accordance with the method of the invention additional sensor structures are used to monitor the person's back position. For example, an optical sensor or camera monitors a distance of the person's back relative to the back of a chair, a pressure sensor or flex sensor cushion that monitors contact of the person's back with the back of the chair and/or a stretch sensor or accelerometer that monitor the position of the person's back while the person is sitting. Regardless of which sensor or set of sensors are used, all of the sensors provide feedback related to the person's posture to a detecting unit and a computing unit generates output signals that are representative of the person's posture.

### BRIEF DESCRIPTION OF THE DRAWINGS

FIG. 1 illustrates a view of a spine with contact zones, in accordance with the embodiments of the invention.

FIG. 2A illustrates a contact sensor structure with multiple sensing zones, in accordance with the embodiments of the invention.

FIG. 2B illustrates a contoured pad with a contact sensor embedded therein, in accordance with the embodiments of the invention.

FIG. 2C illustrates a cross-sectional view of a sensor structure or sensor strip with electrically coupled opposed flex sensors, in accordance with the embodiments of the invention.

FIG. 2D illustrates a top view of a sensor structure or sensor strip with electrically coupled opposed flex sensors, in accordance with the embodiments of the invention.

FIG. 2E illustrates a posture monitoring device with a sensor structure or sensor strip covered by a protective sleeve, in accordance with the invention.

FIG. 3A illustrates a system for monitoring posture that includes a portable cushion with a sensor structure imbedded therein, in accordance with the embodiments of the invention.

FIG. 3B illustrates a vest with an integrated contact sensor structure, in accordance with the invention.

FIG. 3C illustrates a chair with a back rest cushion that includes an integrated contact sensor structure, in accordance with the invention.

FIGS. 4A-B illustrate a pelvic girdle portion of a human skeleton.

FIG. 4C illustrates a system with a contact sensor structure for measuring a distance between the Ischium protrusions of a person sitting and an optical sensor for measuring a position the person back while the person is sitting, in accordance with the invention.

FIG. 4D illustrates a graphical representation of the measured distance between the Ischium protrusions and the measured position the person's back while the person is sitting, in accordance with the invention.

FIG. 5 illustrates a chair with multiple contact sensor structures and an optical sensor, in accordance with the invention.

FIGS. 6A-B show a forward leaning posture position and a corresponding graphical representation generated by a sensor structure with a pair of opposed flex strips, in accordance with the embodiments of the invention.

FIGS. 7A-B show a straight posture position and a corresponding graphical representation generated by a sensor structure with a pair of opposed flex strips, in accordance with the embodiments of the invention.

FIGS. 8A-B show a backward leaning posture position and a corresponding graphical representation generated by a sensor structure with pair of opposed flex strips, in accordance with the embodiments of the invention.

### DETAILED DESCRIPTION OF THE INVENTION

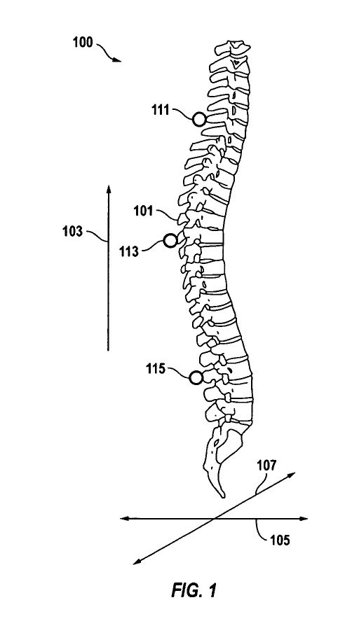

The present invention is directed to a system for monitoring posture. FIG. 1 illustrates a view 100 of a spine 101 with multiple zones or points 111, 113 and 115. The system of the present invention includes a sensor structure that is placed in contact communication with a person's body at multiple contact positions corresponding to, for example, the multiple zones or points 111, 113 and 115 shown. The system of the present invention is capable of measuring or monitoring curvature at the multiple zones or points in one or more directions as represented by the arrows 103, 105 and 107.

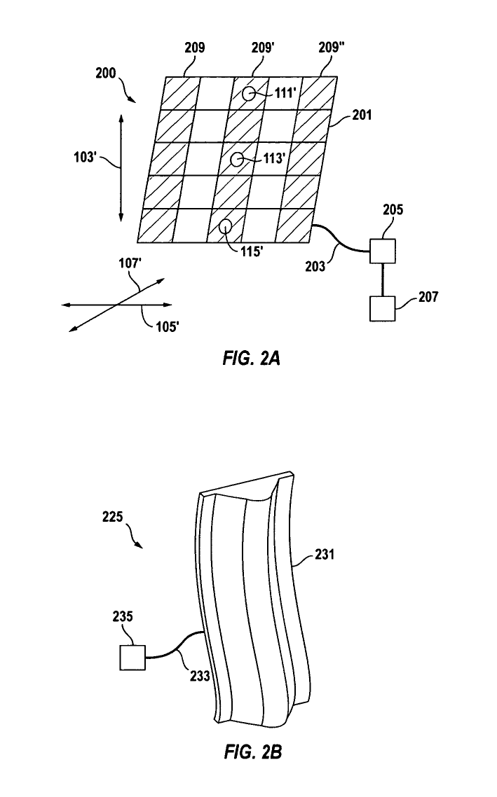

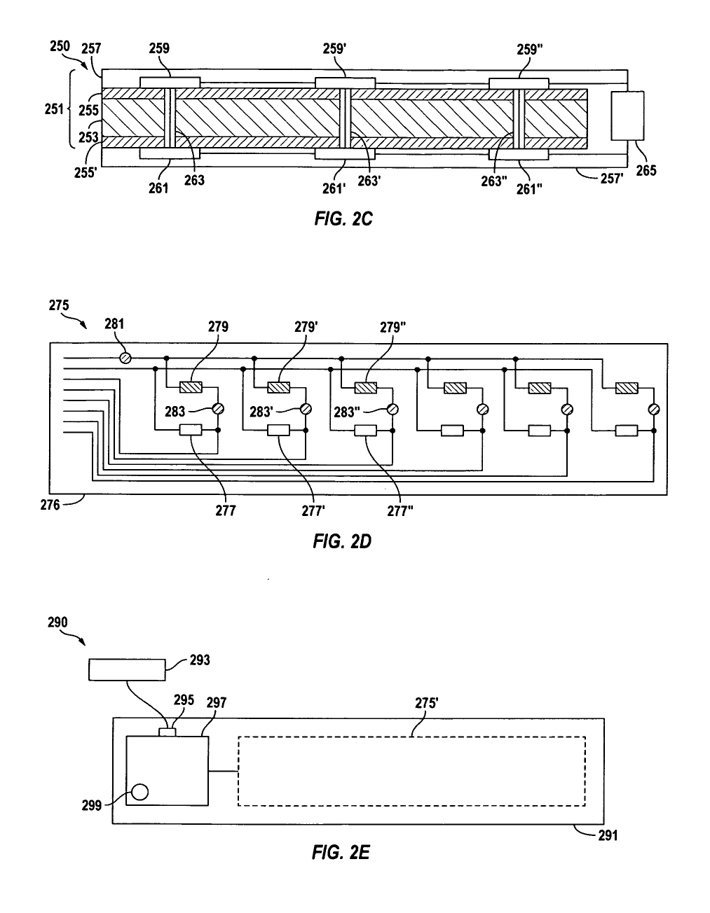

FIG. 2A illustrates a view 200 of a contact sensor structure 201. Preferably, the sensor structure 201 includes one or more sensors, sensor sheets or zones 209, 209′ and 209″ that have one or more electrical properties that change in response to structural or geometrical deformations. The sensors, sensor sheets or zones 209, 209′ and 209″ are also referred to herein as contact sensors. The contact sensors 209, 209′ and 209″ are formed from flex sensors, stretch sensors and pressure sensors, such as described above. The contact sensors 209, 209′ and 209″ are configured for being placed in physical communication with multiple zones or points 111′, 113′ and 115′ and measuring changes in curvatures at the multiple zones or points 111′, 113′ and 115′ in one or more directions as indicated by the arrows 103′, 105′ and 107′. The measured changes in curvatures at the multiple zones or points 111′, 113′ and 115′ are then used by the system to compute, map or monitor posture of a portion of a persons body.

Still referring to FIG. 2A, the sensor structure 201 and each of the contact sensors 209, 209′ and 209″ are electrically coupled to a detecting unit 205 through one or more electrical connections 203. The detecting unit 205 includes all of the necessary circuitry for measuring or monitoring changes in conductance or resistance of each of the contact sensors 209, 209′ and 209″ that result from structural or geometrical deformations at the multiple zones or points 111′, 113′ and 115′. The detecting unit 205 generates electrical signals corresponding to the structural or geometrical deformations and communicates the electrical signals to a computing unit 207 by an electrical connection or by a wireless transmitter (not show). The computing unit 207 is, for example, a personal computer, a smart phone, an on-board vehicle computer, or other computing device with a micro-processor and memory. The computing unit 207 receives the electrical signals from the detecting unit 205 and runs software or firm-ware that processes or analyzes the electrical signals. The computing unit 207 then generates a representation of the posture of the portion of a person's body that is in physical contact with the sensor structure 201. The generated representation of the posture of a portion of a person's body is, for example, in the form of audio signals, tactile signals and/or visual signals.

FIG. 2B shows a view 225 of a contoured sensing pad 231. In accordance with the embodiments the invention the sensor structure 201 and a detecting unit 205 (FIG. 2A) are embedded within the contoured sensing pad 231. In use, the contoured pad 231 is fitted with features that allows the contoured sensing pad 231 to be placed against a portion of a person's body, such as the spinal region of the person's back, shown in FIG. 1. The detecting unit 205 then monitors changes in conductance or resistance in the contact sensors 209, 209′ and 209″ of the sensor structure 201 (FIG. 2A) that result from structural or geometrical deformations at the multiple zones or points 111′, 113′ and 115′ and generates electrical signals. As described above, the detecting unit 205 is configured to be in electrical communication with a computing unit 255 via a connection hardware or by a wireless transmitter that transmits the electrical signals to the computing unit 255. The computing unit 255 then analyzes the electrical signals from the detecting unit 205 to generate output signals corresponding to the posture of a portion of the person's body that is in physical contact with the contoured sensing pad 231.

FIG. 2C illustrates a cross-sectional view 250 of a sensor structure or sensor strip 251 that includes opposed sets of electrically coupled flex sensors 259, 259′, 259″ and 261, 261′, 261″. The sets of flex sensors 259, 259′, 259″ and 261, 261′, 261″ are electrically coupled together by leads that pass through apertures 263, 263′, 263″ in the substrate 253. The substrate 253 is preferably a flexible plastic material of film, such as a polyester film. The substrate 253 is coated with any suitable material or materials 255 and 255′ to provide protect layers and/or improve adhesion of the sets of flex sensors 259, 259′, 259″ and 261, 261′, 261″ on to the substrate 253. Also, the sets of flex sensors 259, 259′, 259″ and 261, 261′, 261″ can be laminated or coated with layers of protective materials 257 and 257′ to electrically isolate and protect the flex sensors 259, 259′, 259″ and 261, 261′, 261″ from the environment. The sensor structure or sensor strip 251 includes a voltage bridge 265 that is electrically coupled to the flex sensors 259, 259′, 259″ and 261, 261′, 261″ to monitor and measure structural or geometrical deformations in the sensor structure or sensor strip 251, such as described in detail above and below.

FIG. 2D illustrates a top view of a sensor structure or sensor strip 275 with electrically coupled opposed sets flex sensors 277, 277′ 277″ and 279, 279′, 279″. The flex sensors 277, 277′ 277″, represented by clear boxes, are flex sensors on the front side of a substrate structure 276 and the flex sensors 279, 279′, 279″, represented by the hatched boxes, are flex sensors on the back side of the substrate structure 276. Again the flex sensors 277, 277′ 277″ and 279, 279′, 279″ are electrically couple together through leads that pass through the substrate structure 276 via apertures 281, 283, 283′ and 283″. The flex sensors 277, 277′ 277″ and 279, 279′, 279″ are connected to electrical components of a detecting unit through any suitable connector or connectors. Electrical components include, but are not limited to, an analog to digital converter, a voltage divider, a processing or computing unit and a wireless transmitter.

FIG. 2E illustrates a posture monitoring device 290 with a sensor structure or sensor strip 275′ covered by a protective sleeve 291. The posture monitoring device 290 preferably includes a 3-axis solid state accelerometer 299 that is coupled to a detecting unit 297 for monitoring changes in position of the posture monitoring device 290. Electronics within the detecting unit 297 are configured to generate electrical signals from structural or geometrical deformations that detected by sensor structure or sensor strip 275′ and/or changes in position detected by the 3-axis solid state accelerometer 299. Preferably, the electronics 297 include a connector 295 for electrically coupling the detecting unit 297 to a periphery electronic device, such as a computer, a smart phone or any other suitable electronic device capable of generating an audio and/or graphical representation from the electrical signals. The sensor structure or sensor strip 275′ preferably includes at least one pair of opposed flex sensors, similar to the opposed pairs of flex sensors describe above with reference to FIGS. 2C and 2D.

In accordance with the embodiment of the invention, flex sensors are printed or coated onto a suitable flexible substrate, such as described above with reference to FIG. 2C. During a drying process of the flex sensors, the electrical sensitivity of the flex sensors can be enhanced by what is what is referred to herein as stress drying. In the stress drying process, a flexible substrate and a flex sensor deposited thereon are bent during the drying process. This stress drying process appears increase structure defects in the flex sensor structure and increases the electrical response of the flex sensors to subsequent structural or geometric deformations.

In accordance with the embodiments of the invention a sensor structure is formed by depositing at least one set of flex sensors on opposite sides of a flexible substrate and electrically coupling the opposed sets of flex sensor through, for example apertures on the flexible substrate, to provide a voltage bridge that cancels noise and provide a contact sensor that measures both concave and convex changes in curvature. Each or one of the opposed flex sensors can include staggered layers of flex sensing material with multiple contacts to provide multiple sensing zones on the sensor structure.

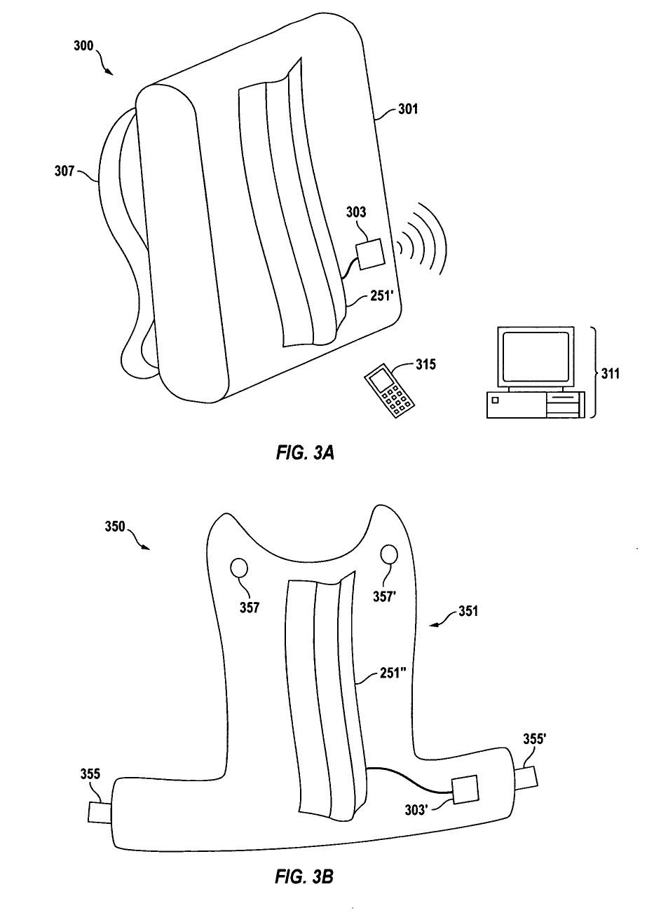

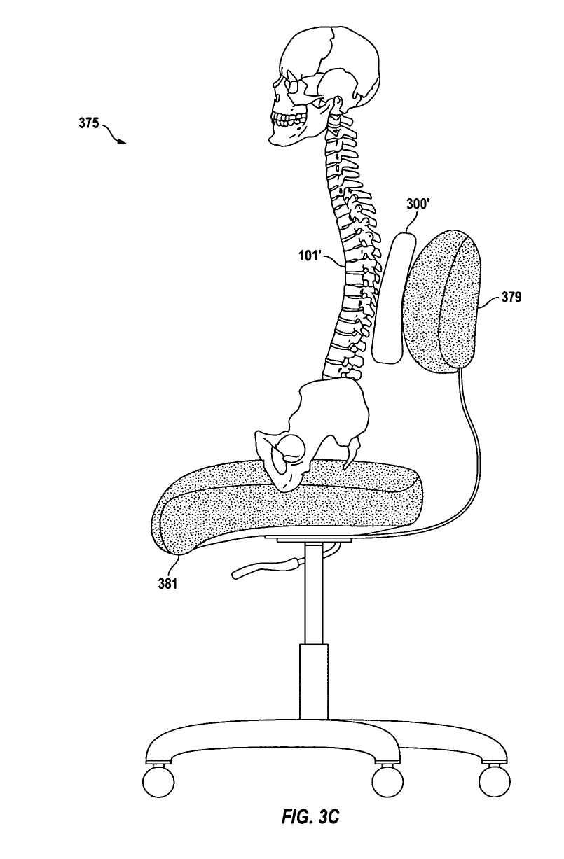

FIG. 3A illustrates a system 300 for monitoring posture that includes a portable cushion 301 with a sensor structure 251′ embedded therein. The portable cushion 301 includes, for example, a strap 307 to attached to a back of a chair and/or a car seat. The system 300 also includes a detecting unit 303 embedded in the cushion structure 301 that includes a wireless transmitter. Where the detecting unit 303 includes a wireless transmitter, the wireless transmitter is preferably a radio transmitter that transmits over remote network or is a radio transmitter that transmits in a peer-to peer network. The wireless transmitter broadcasts electrical signals from the detecting unit 303 to a computing unit such as a personal commuter 311 or a smart phone 315. The personal computer 311 or the smart phone 315 analyzes the electrical signals from the detecting unit 303 to generate output signals corresponding to the posture of a portion of a person's body in physical contact with the portable cushion 301.

In accordance with further embodiments of the invention a system includes a sensor structure with multiple sensing zones that is integrate into an article of clothing. For example, a sensor structure is integrated into a belt, a vest, a band, a belt, a brace sportswear and/or footwear. FIG. 3B illustrates a view 350 of a vest 351 with an integrated sensor structure 251″. Again the sensor structure 251″ includes contact sensors that monitor posture of a portion of a person's body through physical communication with the sensor structure 251″. The vest 351 also includes a detecting unit 303′ that transmits electrical signals to a computing unit (not shown) for generating output signals corresponding the posture of a portion of the persons body in physical contact with the sensor structure 251″. The vest 351 includes, for example, clips or buckles 355 and 355′ for securing the vest around a torso of the person. In yet further embodiments of the invention, in addition to the sensor structure 251″ the vest 351 includes one or more accelerometer sensors 357 and 357′ that are in communication the detecting unit 303′. In operation, the one or more accelerometers sensors 357 and 357′ communicates an orientation or position of the person wearing the vest 351 to a computing unit through the detecting unit 303′. The measured orientation or position of the person is then used to further generate output signals corresponding to posture of the person wearing the vest 351.

FIG. 3C illustrates a chair 375 with a back rest cushion 379 that includes a contact sensor 300′ attached thereto. In operation, a person, represented by the skeletal structure 101′, sits on a seat portion 381 of the chair 375. When the person 101′ leans against the contact sensor 300′ with multiple contact sensor zones, the contact sensor 300′ monitors and maps the person's posture. The person's posture is communicated from a detecting unit to a computing unit (not shown), such as described previously with respect to FIGS. 2A-B and FIGS. 3A-B.

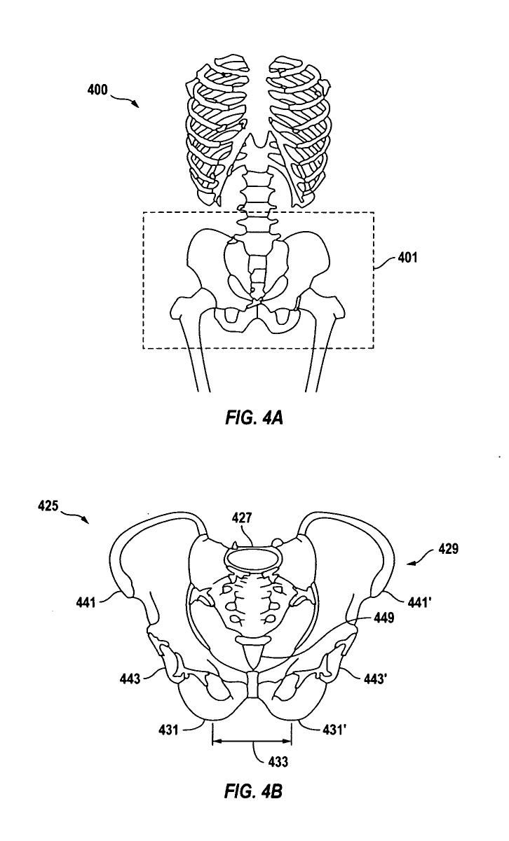

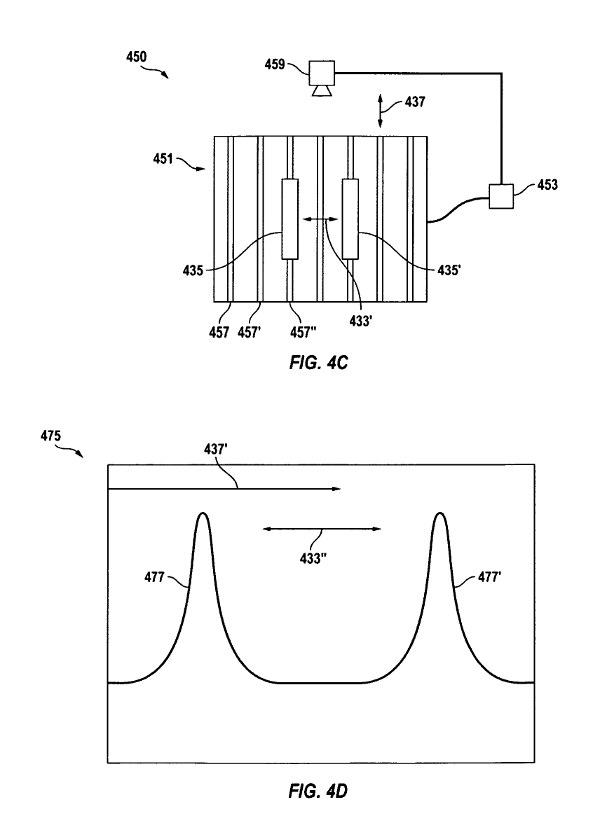

FIG. 4A illustrates lower portion 400 of a human skeleton with the pelvis portion contained within the box 401. Referring to FIG. 4B showing a view 425 of a pelvic girdle 429. The pelvic girdle 429 includes sacrum 427 and Coccyx (tail bone) 449 which are seated or cradled within the Iliac crest. The pelvis includes several bones, the largest of which are the Ilium bones 441 and 441′ which from the Iliac crest. The smaller Acetabulum bones 443 and 443′ and Ischium protrusions 431 and 431′ form the lower portion of the pelvis; humans sit on the Ischium protrusion 431 and 431′ of the pelvis 429. The sacrum 427 pivots in between the Iliac crest and depending on the position of sacrum 427, the distance between the Ischium protrusions 431 and 431′ changes as indicated by the arrow 433. Because the position of the sacrum 427 is correlated to the posture of the spine of a person siting, the distance 433 between the Ischium protrusions 431 and 431′ provides an indirect measurement of the posture of the person sitting.

FIG. 4C illustrates a schematic representation of a system 450 for monitoring a person's posture while sitting by measuring a distance 433′ between the Ischium protrusions 431 and 431 (FIG. 4B) with a contact sensor 451. The contact sensor 451 includes several sensing zones 457, 457′ and 457″, which allow the contact sensor to map the positions 435 and 435′ of the Ischium protrusions 431 and 431 while the person is sitting. The system 450 includes a detecting unit and a processing unit, represented by the box 453, that detects changes in an electrical property of each of the sensing zones 457, 457′ and 457″ caused by physical communication of the Ischium protrusions 431 and 431 with the sensing zones 457, 457′ and 457″. The detecting unit and a processing unit 453 then generates a representation of the person's posture while the person is sitting.

Still referring to FIG. 4C, in further embodiments of the invention the system 450 includes an optical detector 459, such as a camera, that measures a distance, represented by the arrow 437, or position of the person's back while the person is sitting. The optical sensor 459 is in electrical communication with the detecting unit and the processing unit 453 and provides additional data to monitor and evaluate the posture of the person while the person is sitting.

FIG. 4D illustrates a graphical representation 475 of a measured distance 433″ between Ischium protrusions, represented by the peaks 477 and 477′ and the measured distance 437′ of the person back by the optical sensor 459. Using these two parameters, the system 450 (FIG. 4C) can determine if the person's sitting posture is optimized.

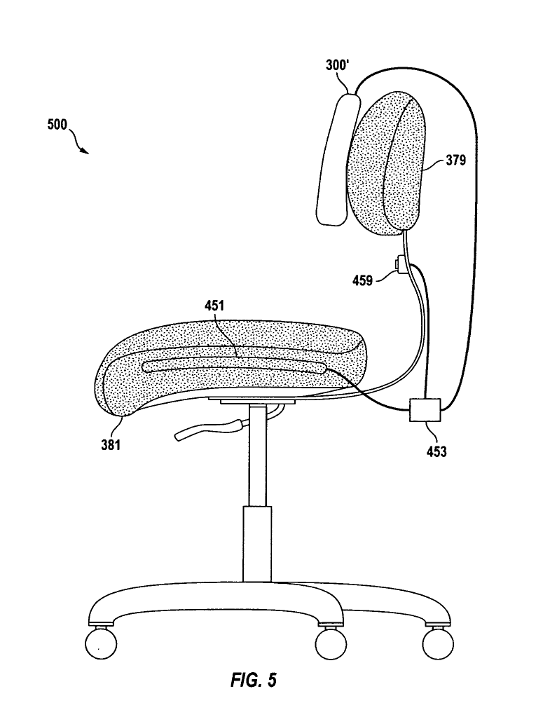

FIG. 5 illustrates a system for monitoring posture that includes a chair 500 with multiple contact sensors 300′ and 451 and an optical sensor 459. In accordance with the embodiments of the invention the contact sensor 451 is embedded within the seat cushion 381 of the chair 500 and the contact sensor 300 is a portable cushion that attaches to the back rest 379 of the chair 500, similar to the portable cushion 301 described with reference to FIG. 3A. The optical sensor 459 monitors the location or position of the person's back, such as described above, while the contact sensors 451 and 300″ map contact positions of the person's body while the person is sitting. The contact sensors 451 and 300′ and the optical sensor 459 are all in electrical communication with a detecting unit and processing unit 453 that generates a representation of the person's posture while the person is sitting.

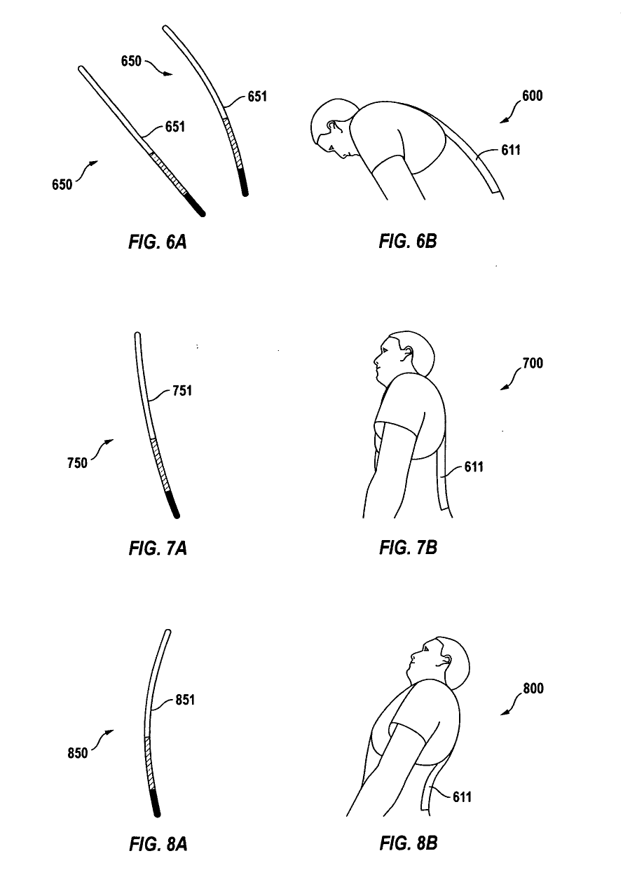

FIG. 6A shows a representation 600 of a person 601 wearing a posture detection device 611 of the present invention with a forward leaning posture position. The posture detection device 611 is integrated into an article of sports clothing and includes a set of opposed flex sensors, electronics and a wireless transmitter. The electrical signals from the electronics of the posture detection device 611 are transmitted via the wireless transmitter to a computer (not shown). The computer, along with the appropriate software, generates a graphical representation 650 of the forward leaning postures of the person 601 in the form of a curve 651, as shown in FIG. 6B.

FIG. 7A shows a representation 700 of a person 701 wearing the posture detection device 611 of the present invention with an erect posture position, wherein the electrical signals from the electronics of the posture detection device 611 have been transmitted via the wireless transmitter to a computer to generate a graphical representation 750 of the erect posture of the person 701 in the form of a curve 751, as shown in FIG. 7B.

FIG. 8A shows a representation 800 of a person 801 wearing the posture detection device 611 of the present invention with a backward leaning posture position. As described above, the electrical signals from the electronics of the posture detection device 611 has been transmitted via the wireless transmitter to a computer to generate a graphical representation 850 of the backward leaning posture of the person 801 in the form of a curve 851, as shown in FIG. 8B.

In accordance with this embodiment described above, the posture detection device 611 includes a contact sensing strip. The contact sensing strip is formed by depositing multiple staggered and continuous layers of flex sensors onto a flexible substrate. The contact sensing strip is electrically couple to an opposed flex strip deposited on the opposite side of the flexible substrate through leads that pass through apertures, such as described above with reference to FIGS. 2C and 2D. As a result, the contact sensing strip has multiple detection zones or multiple sensing zones as indicated by the hatched regions on the curves 651, 751 and 851. It can be seen from FIGS. 6A-B, 7A-B and 8A-B that the posture detection device 611 of the present invention is capable of being integrated into any suitable article of clothing to monitor posture of a person wearing the article of clothing.

The present invention has been described in terms of specific embodiments incorporating details to facilitate the understanding of the principles of construction and operation of the invention. As such, references, herein to specific embodiments and details thereof are not intended to limit the scope of the claims appended hereto. It will be apparent to those skilled in the art that modifications can be made in the embodiments chosen for illustration without departing from the spirit and scope of the invention.

What is claimed is:

1. An system for monitoring posture comprising:
a) a first sensor structure with multiple contact sensors for placing in contact communication with a portion of a persons body, wherein an electrical property of the multiple contact sensors is responsive to geometric deformations;
b) a detecting unit in electrical communication with the multiple contact sensors that monitors changes in the electrical property of the multiple contact sensors and generates electrical signals corresponding to the geometric deformations in the multiple contact sensors; and
c) a computing unit in communication with the detecting unit that receives the electrical signals and that generates output signals corresponding to a posture of the portion of the person's body based on the electrical signals.

2. The system of claim 1, wherein the multiple contact sensors include a piezo-resistive sensor sheets.

3. The system of claim 1, wherein the multiple contact sensors include a resistive ink sensor sheets.

4. The system of claim 1, wherein the first sensor structure includes a cushion and wherein the multiple contact sensors are imbedded within the cushion.

5. The system of claim 1, further comprising a second sensor structure, wherein the second sensor structure includes a sensor selected from the group consisting of an optical sensor, an accelerometer sensor, a pressure sensor, a stretch sensor and a flex sensor.

6. The system of claim 5, wherein the first sensor structure and second sensor structure are integrated into an article of furniture.

7. The system of claim 1, wherein the first sensor structure is attached to an article of clothing.

8. The system of claim 7, wherein the article of clothing is selected from the group consisting of a belt, a vest, a band, a brace and foot ware or shoe.

9. The system of claim 1, wherein the computing unit is in communication with the detecting unit via a wireless transmitter.

10. The system of claim 1, wherein the computing unit includes a micro-processor for running software that generates a graphical representation of the posture of person's body from the output signals.

11. A system for monitoring posture comprising:
a) a cushion structure;
b) a sensor structure with multiple contact sensors that have an electrical property that is response to mechanical deformations and that are embedded within the cushion structure;
c) a circuit for measuring changes in the electrical property of the multiple contact sensors; and
d) a computer for generating representations of posture of a portion of a user's body that is in contact communication with the cushion structure based on the measured changes in the electrical property of the sensor structure.

12. The system of claim 11, wherein the multiple contact sensors include one or more pressure sensors or flex sensors.

13. The system of claim 12, wherein the electrical property is resistance or conductance.

14. The system of claim 11, wherein the cushion structure is integrated in a furniture article.

15. The system of claim 11, further comprising a second sensor structure, wherein the second sensor structure includes one or more optical sensors, accelerometer sensors, pressure sensors, stretch sensors and flex sensors.

16. The system of claim 11, wherein cushion structure is integrated into an article of clothing.

17. An system for monitoring posture comprising:
a) a sensor strip with at least one set of flex sensors on opposite sides of a flexible substrate that are electrically coupled to provide a voltage bridge, wherein the sensor strip is responsive to structural or geometrical deformations;
b) a detecting unit in electrical communication with the sensor strip that monitors changes in the electrical property of the sensor strip and generates electrical signals corresponding to the structural or geometrical deformations; and
c) a computing unit in communication with the detecting unit that receives the electrical signals and generates output signals corresponding to a posture of the portion of the person's body that is in physical communication with the sensor strip based on the electrical signals.

18. The system of claim 17, further comprising and accelerometer in communication with the detecting unit for measuring an orientation or position of a person wearing the system.

19. The system of claim 17, wherein the detecting unit includes a wireless transmitter for transmitting the electrical signals corresponding to the structural or geometrical deformations to the computing unit.

20. The system of claim 17, wherein the sensor strip is attached to an article of clothing.

21. The system of claim 17, wherein the sensor strip has multiple sensing zones.
